(19) United States
(12) Patent Application Publication
(10) Pub. No.: US 2014/0275889 A1
(43) Pub. Date: Sep. 18, 2014

(54) SYSTEMS AND METHODS FOR DETERMINING RESPIRATION INFORMATION FROM SEGMENTS OF A PHOTOPLETHYSMOGRAPH

(71) Applicant: Covidien LP, Mansfield, MA (US)

(72) Inventors: Paul Stanley Addison, Edinburgh (GB); James Watson, Dunfermline (GB)

(21) Appl. No.: 13/841,387

(22) Filed: Mar. 15, 2013

(51) Int. Cl. A61B 5/00; A61B 5/1455; A61B 5/0205

(52) U.S. Cl. CPC A61B 5/7278; A61B 5/0205; A61B 5/14551; A61B 5/0816
USPC 600/324

(57) ABSTRACT

A physiological monitoring system may determine respiration information from a PPG signal. The system may analyze the PPG signal with respect to itself by associating values of the PPG signal with values of a time-delayed version of the PPG signal to create pairs of associated values. A subset of associated values may be identified. Respiration metric values may be determined based on the subset of pairs. The respiration metric values may be amplitude values and/or time values corresponding to the subset of pairs. The respiration metric values may be analyzed using autocorrelation, cross-correlation, or other signal processing techniques to determine respiration information such as respiration rate.

The present disclosure relates to physiological signal processing, and more particularly relates to determining respiration information from a physiological signal.

### SUMMARY

A physiological monitoring system may be configured to determine respiration information from a physiological signal by analyzing the physiological signal with respect to a time-delayed version of itself. Values of the physiological signal may be associated with values of a time-delayed version of the same signal in order to form pairs of associated values. The pairs of associated values may be analyzed to identify a subset of pairs, from which respiration information is determined.

In some embodiments, the subset of pairs may be identified by considering the pairs of associated values in a two-dimensional space and identifying pairs of associated values that correspond to a curve. In some embodiments, the subset of pairs may be identified by determining angles corresponding to the pairs of associated values and identifying pairs of associated values whose angles correspond to a predetermined angle. In some embodiments, the subset of pairs may be identified by identifying zero crossings associated with the pairs of associated values.

Respiration metric values may be determined based on the subset of pairs and the respiration metric values may be used to determine respiration information. In some embodiments, the respiration metric values may be amplitude values associated with the subset of pairs. In some embodiments, the respiration metric values may be time values associated with the subset of pairs. The respiration metric values may be processed using autocorrelation, cross-correlation, any other suitable signal processing technique, or any combination thereof to determine the respiration information.

### BRIEF DESCRIPTION OF THE FIGURES

The above and other features of the present disclosure, its nature and various advantages will be more apparent upon consideration of the following detailed description, taken in conjunction with the accompanying drawings in which:

FIG. 1 shows an illustrative patient monitoring system in accordance with some embodiments of the present disclosure;

FIG. 2 is a block diagram of the illustrative patient monitoring system of FIG. 1 coupled to a patient in accordance with some embodiments of the present disclosure;

FIG. 3 shows a block diagram of an illustrative signal processing system in accordance with some embodiments of the present disclosure;

FIG. 4 shows an illustrative PPG signal that may be analyzed in accordance with some embodiments of the present disclosure;

FIG. 5A shows an illustrative PPG signal that may be analyzed in accordance with some embodiments of the present disclosure;

FIG. 5B shows an illustrative processed PPG signal in accordance with some embodiments of the present disclosure;

FIG. 6 shows an illustrative attractor generated from pairs of associated values of a processed PPG signal in accordance with some embodiments of the present disclosure;

FIG. 7A shows an illustrative plot of amplitude values in accordance with some embodiments of the present disclosure;

FIG. 7B shows an illustrative plot of time values in accordance with some embodiments of the present disclosure;

FIG. 8A shows an illustrative plot of respiration metric values in accordance with some embodiments of the present disclosure;

FIG. 8B shows an illustrative plot of a correlation signal generated in accordance with some embodiments of the present disclosure;

FIG. 9 is a flowchart showing illustrative steps for determining respiration information in accordance with some embodiments of the present disclosure; and

FIG. 10A-C are flowcharts showing illustrative steps for determining respiration information based on respiration metrics in accordance with some embodiments of the present disclosure.

### DETAILED DESCRIPTION OF THE FIGURES

The present disclosure is directed towards determining respiration information based on segments of a physiological signal. A patient monitoring system may receive one or more physiological signals, such as a photoplethysmograph (PPG) signal generated by a pulse oximeter sensor coupled to a subject. The patient monitoring system may condition (e.g., amplify, filter, sample, digitize) the received physiological signals before determining the respiration information.

The patient monitoring system may determine respiration information by associating values of the physiological signal with values of a time-delayed version of the same signal to form pairs of associated values. The term attractor, as used herein, is used to refer to the pairs of associated values when considered in two-dimensional space. For example, the term attractor is used to refer to a plot of the pairs of associated values. The pairs of associated values may be analyzed to identify a subset of pairs. The subset of pairs may be identified by considering the pairs of associated values in a two-dimensional space, where the subset of pairs approximately forms a curve (e.g., a straight line). In some embodiments, this may be accomplished graphically, for example, by plotting an attractor and identifying portions of the attractor that intersect a curve, or computationally. This may be referred to as taking a slice of the attractor. In some embodiments, the subset of pairs may also be identified by determining angles corresponding to the pairs of associated values and identifying pairs of associated values whose angles correspond to a predetermined angle. In some embodiments, the subset of pairs may be identified by identifying zero crossings associated with the pairs of associated values.

The subset of pairs may be used to determine one or more respiration metrics from which respiration information may be determined. For example, the subset of pairs may be processed to determine amplitude values associated with the subset of pairs. The amplitude values may be calculated, for example, as the distances from an origin to the subset of pairs, when the pairs are considered in two-dimensional space. As another example, the subset of pairs may be used to determine time values associated with the subset of pairs. The time values may be calculated as time differences determined from adjacent ones of the subset of pairs. For example, a time associated with one pair may be subtracted from a time associated with the adjacent pair. The time differences may also be determined graphically, for example, by examining the properties of a plotted attractor where it intersects a curve. The one or more respiration metrics (e.g., amplitudes values and/or time values) may be analyzed using one or more techniques (e.g., an autocorrelation technique, a cross-correlation technique, any other suitable techniques, or any combination thereof) to determine respiration information.

One type of medical device that may be used to determine respiration information in accordance with the present disclosure is an oximeter. An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient). Pulse oximeters may be included in patient monitoring systems that measure and display various blood flow characteristics including, but not limited to, the oxygen saturation of hemoglobin in arterial blood. Such patient monitoring systems may also measure and display additional physiological parameters, such as a patient's pulse rate and respiration information.

An oximeter may include a light sensor that is placed at a site on a patient, typically a fingertip, toe, forehead or earlobe, or in the case of a neonate, across a foot. The oximeter may use a light source to pass light through blood perfused tissue and photoelectrically sense the absorption of the light in the tissue. In addition, locations that are not typically understood to be optimal for pulse oximetry serve as suitable sensor locations for the monitoring processes described herein, including any location on the body that has a strong pulsatile arterial flow. For example, additional suitable sensor locations include, without limitation, the neck to monitor carotid artery pulsatile flow, the wrist to monitor radial artery pulsatile flow, the inside of a patient's thigh to monitor femoral artery pulsatile flow, the ankle to monitor tibial artery pulsatile flow, and around or in front of the ear. Suitable sensors for these locations may include sensors for sensing absorbed light based on detecting reflected light. In all suitable locations, for example, the oximeter may measure the intensity of light that is received at the light sensor as a function of time. The oximeter may also include sensors at multiple locations. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate any of a number of physiological parameters, including an amount of a blood constituent (e.g., oxyhemoglobin) being measured as well as a physiological rate (e.g., pulse rate and respiration rate) and when each individual pulse or breath occurs.

In some applications, the light passed through the tissue is selected to be of one or more wavelengths that are absorbed by the blood in an amount representative of the amount of the blood constituent present in the blood. The amount of light passed through the tissue varies in accordance with the changing amount of blood constituent in the tissue and the related light absorption. Red and infrared (IR) wavelengths may be used because it has been observed that highly oxygenated blood will absorb relatively less Red light and more IR light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based at least in part on Lambert-Beer's law. The following notation will be used herein:

I(λ,t)=I_{O}(λ)exp(−(sβ_{O}(λ)+(1−s)βᵣ(λ))l(t)) (1)

where:
λ=wavelength;
t=time;
I=intensity of light detected;
I₀=intensity of light transmitted;
s=oxygen saturation;
β₀,βᵣ=empirically derived absorption coefficients; and
l(t)=a combination of concentration and path length from emitter to detector as a function of time.

The traditional approach measures light absorption at two wavelengths (e.g., Red and IR), and then calculates saturation by solving for the “ratio of ratios” as follows.

1. The natural logarithm of Eq. 1 is taken (“log” will be used to represent the natural logarithm) for IR and Red to yield

log I=log I₀−(sβ₀+(1−s)βᵣ) (2)

2. Eq. 2 is then differentiated with respect to time to yield

$$\frac{d log I}{d t} = - ( s {β}_{O} + ( 1 - s ) {β}_{r} ) \frac{d l}{d t} . \qquad ( 3 )$$

3. Eq. 3, evaluated at the Red wavelength λ_{R}, is divided by Eq. 3 evaluated at the IR wavelength λ_{IR} in accordance with

$$\frac{d log I ( {λ}_{R} ) / d t}{d log I ( {λ}_{IR} ) / d t} = \frac{s {β}_{O} ( {λ}_{R} ) + ( 1 - s ) {β}_{r} ( {λ}_{R} )}{s {β}_{O} ( {λ}_{IR} ) + ( 1 - s ) {β}_{r} ( {λ}_{IR} )} . \qquad ( 4 )$$

4. Solving for s yields

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\begin{matrix}\frac{d log I ( {λ}_{R} )}{d t} ( {β}_{O} ( {λ}_{IR} ) - {β}_{r} ( {λ}_{IR} ) ) - \\ \frac{d log I ( {λ}_{IR} )}{d t} ( {β}_{O} ( {λ}_{R} ) - {β}_{r} ( {λ}_{R} ) )\end{matrix}} . \qquad ( 5 )$$

5. Note that, in discrete time, the following approximation can be made:

$$\frac{d log I ( λ , t )}{d t} ≃ log I ( λ , {t}_{2} ) - log I ( λ , {t}_{1} ) . \qquad ( 6 )$$

6. Rewriting Eq. 6 by observing that log A−log B=log(A/B) yields

$$\frac{d log I ( λ , t )}{d t} ≃ log ( \frac{I ( {t}_{2} , λ )}{I ( {t}_{1} , λ )} ) . \qquad ( 7 )$$

7. Thus, Eq. 4 can be expressed as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R , \qquad ( 8 )$$

where R represents the “ratio of ratios.”
8. Solving Eq. 4 for s using the relationship of Eq. 5 yields

$$s = \frac{{β}_{r} ( {λ}_{R} ) - R {β}_{r} ( {λ}_{IR} )}{R ( {β}_{O} ( {λ}_{IR} ) - {β}_{r} ( {λ}_{IR} ) ) - {β}_{O} ( {λ}_{R} ) + {β}_{r} ( {λ}_{R} )} . \qquad ( 9 )$$

9. From Eq. 8, R can be calculated using two points (e.g., PPG maximum and minimum), or a family of points. One method applies a family of points to a modified version of Eq. 8. Using the relationship

$$\frac{d log I}{d t} = \frac{d I / d t}{I} , \qquad ( 10 )$$

Eq. 8 becomes

$$\begin{matrix}\frac{d log I ( {λ}_{R} )}{d t} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R ,\end{matrix} \qquad ( 11 )$$

which defines a cluster of points whose slope of y versus x will give R when

x=[I(t₂,λ_{IR})−I(t₁,λ_{IR})]I(t₁,λ_{R}), (12)

and

y=[I(t₂,λ_{R})−I(t₁,λ_{R})]I(t₁,λ_{IR}) (13)

Once R is determined or estimated, for example, using the techniques described above, the blood oxygen saturation can be determined or estimated using any suitable technique for relating a blood oxygen saturation value to R. For example, blood oxygen saturation can be determined from empirical data that may be indexed by values of R, and/or it may be determined from curve fitting and/or other interpolative techniques.

FIG. 1 is a perspective view of an embodiment of a patient monitoring system 10. System 10 may include sensor unit 12 and monitor 14. In some embodiments, sensor unit 12 may be part of an oximeter. Sensor unit 12 may include an emitter 16 for emitting light at one or more wavelengths into a patient's tissue. A detector 18 may also be provided in sensor unit 12 for detecting the light originally from emitter 16 that emanates from the patient's tissue after passing through the tissue. Any suitable physical configuration of emitter 16 and detector 18 may be used. In an embodiment, sensor unit 12 may include multiple emitters and/or detectors, which may be spaced apart. System 10 may also include one or more additional sensor units (not shown) that may take the form of any of the embodiments described herein with reference to sensor unit 12. An additional sensor unit may be the same type of sensor unit as sensor unit 12, or a different sensor unit type than sensor unit 12. Multiple sensor units may be capable of being positioned at two different locations on a subject's body; for example, a first sensor unit may be positioned on a patient's forehead, while a second sensor unit may be positioned at a patient's fingertip.

Sensor units may each detect any signal that carries information about a patient's physiological state, such as an electrocardiograph signal, arterial line measurements, or the pulsatile force exerted on the walls of an artery using, for example, oscillometric methods with a piezoelectric transducer. According some embodiments, system 10 may include two or more sensors forming a sensor array in lieu of either or both of the sensor units. Each of the sensors of a sensor array may be a complementary metal oxide semiconductor (CMOS) sensor. Alternatively, each sensor of an array may be charged coupled device (CCD) sensor. In some embodiments, a sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier. It will be understood that any type of sensor, including any type of physiological sensor, may be used in one or more sensor units in accordance with the systems and techniques disclosed herein. It is understood that any number of sensors measuring any number of physiological signals may be used to determine physiological information in accordance with the techniques described herein.

In some embodiments, emitter 16 and detector 18 may be on opposite sides of a digit such as a finger or toe, in which case the light that is emanating from the tissue has passed completely through the digit. In some embodiments, emitter 16 and detector 18 may be arranged so that light from emitter 16 penetrates the tissue and is reflected by the tissue into detector 18, such as in a sensor designed to obtain pulse oximetry data from a patient's forehead.

In some embodiments, sensor unit 12 may be connected to and draw its power from monitor 14 as shown. In another embodiment, the sensor may be wirelessly connected to monitor 14 and include its own battery or similar power supply (not shown). Monitor 14 may be configured to calculate physiological parameters (e.g., pulse rate, blood oxygen saturation, and respiration information) based at least in part on data relating to light emission and detection received from one or more sensor units such as sensor unit 12 and an additional sensor (not shown). In some embodiments, the calculations may be performed on the sensor units or an intermediate device and the result of the calculations may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. In some embodiments, the system 10 includes a stand-alone monitor in communication with the monitor 14 via a cable or a wireless network link.

In some embodiments, sensor unit 12 may be communicatively coupled to monitor 14 via a cable 24. In some embodiments, a wireless transmission device (not shown) or the like may be used instead of or in addition to cable 24. Monitor 14 may include a sensor interface configured to receive physiological signals from sensor unit 12, provide signals and power to sensor unit 12, or otherwise communicate with sensor unit 12. The sensor interface may include any suitable hardware, software, or both, which may allow communication between monitor 14 and sensor unit 12.

Patient monitoring system 10 may also include display monitor 26. Monitor 14 may be in communication with display monitor 26. Display monitor 26 may be any electronic device that is capable of communicating with monitor 14 and calculating and/or displaying physiological parameters, e.g., a general purpose computer, tablet computer, smart phone, or an application-specific device. Display monitor 26 may include a display 28 and user interface 30. Display 28 may include touch screen functionality to allow a user to interface with display monitor 26 by touching display 28 and utilizing motions. User interface 30 may be any interface that allows a user to interact with display monitor 26 (e.g., a keyboard, one or more buttons, a camera, or a touchpad).

Monitor 14 and display monitor 26 may communicate utilizing any suitable transmission medium, including wireless (e.g., WiFi, Bluetooth, etc.), wired (e.g., USB, Ethernet, etc.), or application-specific connections. In an exemplary embodiment, monitor 14 and display monitor 26 may be connected via cable 32. Monitor 14 and display monitor 26 may communicate utilizing standard or proprietary communications protocols, such as the Standard Host Interface Protocol (SHIP) developed and used by Covidien of Mansfield, Mass. In addition, monitor 14, display monitor 26, or both may be coupled to a network to enable the sharing of information with servers or other workstations (not shown). Monitor 14, display monitor 26, or both may be powered by a battery (not shown) or by a conventional power source such as a wall outlet.

Monitor 14 may transmit calculated physiological parameters (e.g., pulse rate, blood oxygen saturation, and respiration information) to display monitor 26. In some embodiments, monitor 14 may transmit a PPG signal, data representing a PPG signal, or both to display monitor 26, such that some or all calculated physiological parameters (e.g., pulse rate, blood oxygen saturation, and respiration information) may be calculated at display monitor 26. In an exemplary embodiment, monitor 14 may calculate pulse rate and blood oxygen saturation, while display monitor 26 may calculate respiration information such as a respiration rate.

FIG. 2 is a block diagram of a patient monitoring system, such as patient monitoring system 10 of FIG. 1, which may be coupled to a patient 40 in accordance with an embodiment. Certain illustrative components of sensor unit 12 and monitor 14 are illustrated in FIG. 2.

Sensor unit 12 may include emitter 16, detector 18, and encoder 42. In the embodiment shown, emitter 16 may be configured to emit at least two wavelengths of light (e.g., Red and IR) into a patient's tissue 40. Hence, emitter 16 may include a Red light emitting light source such as Red light emitting diode (LED) 44 and an IR light emitting light source such as IR LED 46 for emitting light into the patient's tissue 40 at the wavelengths used to calculate the patient's physiological parameters. In some embodiments, the Red wavelength may be between about 600 nm and about 700 nm, and the IR wavelength may be between about 800 nm and about 1000 nm. In embodiments where a sensor array is used in place of a single sensor, each sensor may be configured to emit a single wavelength. For example, a first sensor may emit only a Red light while a second sensor may emit only an IR light. In a further example, the wavelengths of light used may be selected based on the specific location of the sensor.

It will be understood that, as used herein, the term “light” may refer to energy produced by radiation sources and may include one or more of radio, microwave, millimeter wave, infrared, visible, ultraviolet, gamma ray or X-ray electromagnetic radiation. As used herein, light may also include electromagnetic radiation having any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In some embodiments, detector 18 may be configured to detect the intensity of light at the Red and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the Red and IR wavelengths in the patient's tissue 40.

In some embodiments, encoder 42 may contain information about sensor unit 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters.

Encoder 42 may contain information specific to patient 40, such as, for example, the patient's age, weight, and diagnosis. This information about a patient's characteristics may allow monitor 14 to determine, for example, patient-specific threshold ranges in which the patient's physiological parameter measurements should fall and to enable or disable additional physiological parameter algorithms. This information may also be used to select and provide coefficients for equations from which measurements may be determined based at least in part on the signal or signals received at sensor unit 12. For example, some pulse oximetry sensors rely on equations to relate an area under a portion of a PPG signal corresponding to a physiological pulse to determine blood pressure. These equations may contain coefficients that depend upon a patient's physiological characteristics as stored in encoder 42. Encoder 42 may, for instance, be a coded resistor that stores values corresponding to the type of sensor unit 12 or the type of each sensor in the sensor array, the wavelengths of light emitted by emitter 16 on each sensor of the sensor array, and/or the patient's characteristics. In some embodiments, encoder 42 may include a memory on which one or more of the following information may be stored for communication to monitor 14: the type of the sensor unit 12; the wavelengths of light emitted by emitter 16; the particular wavelength each sensor in the sensor array is monitoring; a signal threshold for each sensor in the sensor array; any other suitable information; or any combination thereof.

In some embodiments, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, data output 84, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium that can be used to store the desired information and that can be accessed by components of the system.

In the embodiment shown, a time processing unit (TPU) 58 may provide timing control signals to light drive circuitry 60, which may control when emitter 16 is illuminated and multiplexed timing for Red LED 44 and IR LED 46. TPU 58 may also control the gating-in of signals from detector 18 through amplifier 62 and switching circuit 64. These signals are sampled at the proper time, depending upon which light source is illuminated. The received signal from detector 18 may be passed through amplifier 66, low pass filter 68, and analog-to-digital converter 70. The digital data may then be stored in a queued serial module (QSM) 72 (or buffer) for later downloading to RAM 54 as QSM 72 is filled. In some embodiments, there may be multiple separate parallel paths having components equivalent to amplifier 66, filter 68, and/or A/D converter 70 for multiple light wavelengths or spectra received. Any suitable combination of components (e.g., microprocessor 48, RAM 54, analog to digital converter 70, any other suitable component shown or not shown in FIG. 2) coupled by bus 50 or otherwise coupled (e.g., via an external bus), may be referred to as “processing equipment.”

In some embodiments, microprocessor 48 may determine the patient's physiological parameters, such as SpO₂, pulse rate, and/or respiration information, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to decoder 74. These signals may include, for example, encoded information relating to patient characteristics. Decoder 74 may translate these signals to enable the microprocessor to determine the thresholds based at least in part on algorithms or look-up tables stored in ROM 52. In some embodiments, user inputs 56 may be used to enter information, select one or more options, provide a response, input settings, any other suitable inputting function, or any combination thereof. User inputs 56 may be used to enter information about the patient, such as age, weight, height, diagnosis, medications, treatments, and so forth. In some embodiments, display 20 may exhibit a list of values, which may generally apply to the patient, such as, for example, age ranges or medication families, which the user may select using user inputs 56.

Calibration device 80, which may be powered by monitor 14 via a communicative coupling 82, a battery, or by a conventional power source such as a wall outlet, may include any suitable signal calibration device. Calibration device 80 may be communicatively coupled to monitor 14 via communicative coupling 82, and/or may communicate wirelessly (not shown). In some embodiments, calibration device 80 is completely integrated within monitor 14. In some embodiments, calibration device 80 may include a manual input device (not shown) used by an operator to manually input reference signal measurements obtained from some other source (e.g., an external invasive or non-invasive physiological measurement system).

Data output 84 may provide for communications with other devices such as display monitor 26 utilizing any suitable transmission medium, including wireless (e.g., WiFi, Bluetooth, etc.), wired (e.g., USB, Ethernet, etc.), or application-specific connections. Data output 84 may receive messages to be transmitted from microprocessor 48 via bus 50. Exemplary messages to be sent in an embodiment described herein may include PPG signals to be transmitted to display monitor module 26.

The optical signal attenuated by the tissue of patient 40 can be degraded by noise, among other sources. One source of noise is ambient light that reaches the light detector. Another source of noise is electromagnetic coupling from other electronic instruments. Movement of the patient also introduces noise and affects the signal. For example, the contact between the detector and the skin, or the emitter and the skin, can be temporarily disrupted when movement causes either to move away from the skin. Also, because blood is a fluid, it responds differently than the surrounding tissue to inertial effects, which may result in momentary changes in volume at the point to which the oximeter probe is attached.

Noise (e.g., from patient movement) can degrade a sensor signal relied upon by a care provider, without the care provider's awareness. This is especially true if the monitoring of the patient is remote, the motion is too small to be observed, or the care provider is watching the instrument or other parts of the patient, and not the sensor site. Processing sensor signals (e.g., PPG signals) may involve operations that reduce the amount of noise present in the signals, control the amount of noise present in the signal, or otherwise identify noise components in order to prevent them from affecting measurements of physiological parameters derived from the sensor signals.

FIG. 3 is an illustrative processing system 300 in accordance with an embodiment that may implement the signal processing techniques described herein. In some embodiments, processing system 300 may be included in a patient monitoring system (e.g., patient monitoring system 10 of FIGS. 1-2). Processing system 300 may include signal input 310, pre-processor 312, processor 314, post-processor 316, and output 318. Pre-processor 312, processor 314, and post-processor 316 may be any suitable software, firmware, hardware, or combination thereof for calculating physiological parameters such as respiration information based on an input signal received from signal input 310. For example, pre-processor 312, processor 314, and post-processor 316 may include one or more hardware processors (e.g., integrated circuits), one or more software modules, computer-readable media such as memory, firmware, or any combination thereof. Pre-processor 312, processor 314, and post-processor 316 may, for example, be a computer or may be one or more chips (i.e., integrated circuits). Pre-processor 312, processor 314, and post-processor 316 may, for example, include an assembly of analog electronic components.

In some embodiments, processing system 300 may be included in monitor 14 and/or display monitor 26 of a patient monitoring system (e.g., patient monitoring system 10 of FIGS. 1-2). In the illustrated embodiment, signal input 310 may generate a PPG signal that was sampled and generated at monitor 14, for example at 76 Hz. Signal input 310, pre-processor 312, processor 314, and post-processor 316 may reside entirely within a single device (e.g., monitor 14 or display monitor 26) or may reside in multiple devices (e.g., monitor 14 and display monitor 26).

Signal input 310 may be coupled to pre-processor 312. In some embodiments, signal input 310 may generate PPG signals corresponding to one or more light frequencies, such as a Red PPG signal and an IR PPG signal. In some embodiments, the signal may include signals measured at one or more sites on a subject's body, for example, a subject's finger, toe, ear, arm, or any other body site. In some embodiments, the signal may include multiple types of signals (e.g., one or more of an ECG signal, an EEG signal, an acoustic signal, an optical signal, a signal representing a blood pressure, and a signal representing a heart rate). The signal may be any suitable biosignal or signals, such as, for example, electrocardiogram, electroencephalogram, electrogastrogram, electromyogram, heart rate signals, pathological sounds, ultrasound, or any other suitable biosignal. The systems and techniques described herein are also applicable to any dynamic signals, non-destructive testing signals, condition monitoring signals, fluid signals, geophysical signals, astronomical signals, electrical signals, financial signals including financial indices, sound and speech signals, chemical signals, meteorological signals including climate signals, any other suitable signal, and/or any combination thereof.

Pre-processor 312 may be implemented by any suitable combination of hardware and software. In an embodiment, pre-processor 312 may be any suitable signal processing device and the signal received from signal input 310 may include one or more PPG signals. An exemplary received PPG signal may be received in a streaming fashion, or may be received on a periodic basis as a sampling window (e.g., every 5 seconds). The received signal may include the PPG signal as well as other information related to the PPG signal (e.g., a pulse found indicator, the mean pulse rate from the PPG signal, the most recent pulse rate estimate, an indicator of invalid samples, and an indicator of artifacts within the PPG signal). It will be understood that signal input 310 may include any suitable signal source, signal generating data, signal generating equipment, or any combination thereof to be provided to pre-processor 312. The signal generated by input signal 310 may be a single signal, or may be multiple signals transmitted over a single pathway or multiple pathways.

Pre-processor 312 may apply one or more signal processing operations to the signal received from signal input 310. For example, pre-processor 312 may apply a pre-determined set of processing operations to signal input 310 to produce a signal that may be appropriately analyzed and interpreted by processor 314, post-processor 316, or both. Pre-processor 312 may perform any necessary operations to provide a signal that may be used as an input for processor 314 and post-processor 316 to determine physiological information such as respiration information. Examples include reshaping the signal for transmission, multiplexing the signal, modulating the signal onto carrier signals, compressing the signal, encoding the signal, filtering the signal, low-pass filtering, bandpass filtering, signal interpolation, downsampling of a signal, attenuating the signal, adaptive filtering, closed-loop filtering, any other suitable filtering, and/or any combination thereof. Other signal processing operations may be performed by pre-processor 312 for determining parameters (e.g., pulse rate) and metrics (e.g., respiration metrics, period variability, and amplitude variability) that are used as inputs to determine physiological information. The physiological information may be respiration information, which may include any information relating to respiration (e.g., respiration rate, change in respiration rate, breathing intensity, etc.). Pre-processor 312 may, for example, identify segments of the input signal, form pairs of associated values from the segments, and determine respiration metrics based on the pairs of associated values.

In some embodiments, pre-processor 312 may be coupled to processor 314 and post-processor 316. Processor 314 and post-processor 316 may be implemented by any suitable combination of hardware and software. Processor 314 may receive physiological information and calculated parameters from pre-processor 312. For example, processor 314 may receive respiration metrics for use in determining respiration information. Processor 314 may utilize the received respiration metrics to calculate respiration information. Processor 314 may be coupled to post-processor 316 and may communicate respiration information to post-processor 316. Processor 314 may also provide other information to post-processor 316 such as the signal age related to the signal used to calculate the respiration information, and a time ratio representative of the useful portion of the respiration information signal. Pre-processor 312 may also provide information to post-processor 316 such as period variability, amplitude variability, and pulse rate information. Post-processor 316 may utilize the received information to calculate output respiration information, as well as other information such as the age of the respiration information and status information relating to the respiration information output (e.g., whether a valid output respiration information value is currently available). Post-processor 316 may provide the output information to output 318.

Output 318 may be any suitable output device such as one or more medical devices (e.g., a medical monitor that displays various physiological parameters, a medical alarm, or any other suitable medical device that either displays physiological parameters or uses the output of post-processor 316 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

In some embodiments, all or some of pre-processor 312, processor 314, and/or post-processor 316 may be referred to collectively as processing equipment. For example, processing equipment may be configured to amplify, filter, sample and digitize an input signal and calculate physiological information from the signal.

Pre-processor 312, processor 314, and post-processor 316 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by pre-processor 312, processor 314, and post-processor 316 to, for example, store data relating to input PPG signals, respiration metrics, respiration information, or other information corresponding to physiological monitoring.

It will be understood that system 300 may be incorporated into system 10 (FIGS. 1-2) in which, for example, signal input 310 may be generated by sensor unit 12 (FIGS. 1 and 2) and monitor 14 (FIGS. 1-2). Pre-processor 312, processor 314, and post-processor 316 may each be located in one of monitor 14 or display monitor 26 (or other devices), and may be split among multiple devices such as monitor 14 or display monitor 26. In some embodiments, portions of system 300 may be configured to be portable. For example, all or part of system 300 may be embedded in a small, compact object carried with or attached to the patient (e.g., a watch, other piece of jewelry, or a smart phone). In some embodiments, a wireless transceiver (not shown) may also be included in system 300 to enable wireless communication with other components of system 10 (FIGS. 1-2). As such, system 10 (FIGS. 1-2) may be part of a fully portable and continuous patient monitoring solution. In some embodiments, a wireless transceiver (not shown) may also be included in system 300 to enable wireless communication with other components of system 10. For example, communications between one or more of pre-processor 312, processor 314, and post-processor 316 may be over BLUETOOTH, 802.11, WiFi, WiMax, cable, satellite, infrared, or any other suitable transmission scheme. In some embodiments, a wireless transmission scheme may be used between any communicating components of system 300.

Pre-processor 312 may determine the locations of pulses within a periodic signal (e.g., a PPG signal) using a pulse detection technique. For ease of illustration, the following pulse detection techniques will be described as performed by pre-processor 312, but any suitable processing device may be used to implement any of the techniques described herein.

An illustrative PPG signal 400 is depicted in FIG. 4. Pre-processor 312 may receive PPG signal 400 from signal input 310, and may identify reference points such as local minimum point 410, local maximum point 412, local minimum point 420, local maximum point 422, and local minimum point 430 in PPG signal 400. Pre-processor 312 may pair each local minimum point with an adjacent maximum point. For example, pre-processor 312 may pair points 410 and 412 to identify one segment, points 412 and 420 to identify a second segment, points 420 and 422 to identify a third segment and points 422 and 430 to identify a fourth segment. The slope of each segment may be measured to determine whether the segment corresponds to an upstroke portion of the pulse (e.g., a positive slope) or a downstroke portion of the pulse (e.g., a negative slope) portion of the pulse. A pulse may be defined as a combination of at least one upstroke and one downstroke. For example, the segment identified by points 410 and 412 and the segment identified by points 412 and 430 may define a pulse. Any suitable points (e.g., maxima, minima, zeros) or features (e.g., pulse waves, notches, upstrokes) of a physiological signal may be identified by pre-processor 312 as reference points.

PPG signal 400 may include a dichrotic notch 450 or other notches (not shown) in different sections of the pulse (e.g., at the beginning (referred to as an ankle notch), in the middle (referred to as a dichrotic notch), or near the top (referred to as a shoulder notch)). Notches (e.g., dichrotic notches) may refer to secondary turning points of pulse waves as well as inflection points of pulse waves. Pre-processor 312 may identify notches and either utilize or ignore them when detecting the pulse locations. In some embodiments, pre-processor 312 may compute the second derivative of the PPG signal to find the local minima and maxima points and may use this information to determine a location of, for example, a dichrotic notch. Additionally, pre-processor 312 may interpolate between points in a signal or between points in a processed signal using any interpolation technique (e.g., zero-order hold, linear interpolation, and/or higher-order interpolation techniques). Some pulse detection techniques that may be performed by pre-processor 312 are described in more detail in U.S. Patent Publication No. 2009/0326395, published Dec. 31, 2009, which is incorporated by reference herein in its entirety.

In some embodiments, reference points may be received or otherwise determined from any other suitable pulse detecting technique. For example, pulse beep flags generated by a pulse oximeter, which may indicate when the pulse oximeter is to emit an audible beep, may be received by processor 314, pre-processor 312, post-processor 316, or any combination thereof for processing in accordance with the present disclosure. The pulse beep flags may be used as reference points indicative of the occurrence of pulses in temporally corresponding places in the associated PPG signal.

The pulse information may be used to determine information to assist in the processing of the physiological signals to determine respiration information. For example, the pulse information may be used to determine the pulse rate and the pulse rate may be used to adjust the filtering of the input signal. In some embodiments, an adjustable band-pass filter may be used to filter the input signal around the pulse rate (e.g., from 0.5 times pulse rate to 1.5 times the pulse rate). The filtered signal may then be further processed to determine respiration information.

An additional illustrative PPG signal 500 is depicted in FIG. 5A. PPG signal 500 may correspond to a 45 second segment of a PPG signal. PPG signal 500 experiences changes in morphology based on respiration and other physiological functions. These changes may or may not be apparent by mere observation. Respiration may cause changes in the shape of the pulse over time, as indicated by points 502, 504, and 506. The shape of pulses may become more or less round due to respiration, thereby affecting the prominence of the dichrotic notch and other signal characteristics. Respiration may also cause fluctuations in the frequency and amplitude of pulses in PPG signal 500. These fluctuations may cause baseline shifts in the signal or may cause subtle changes in the timing between fiducial points on individual pulses. An illustrative baseline shift is depicted in segment 510 as a dashed line.

FIG. 5B shows an illustrative processed PPG signal 522 in accordance with some embodiments of the present disclosure. Processed PPG signal 522 may be generated, for example, by pre-processor 312 (FIG. 3). PPG signal 522 may have been processed to minimize undesirable signal components. In some embodiments, processed PPG signal 522 may be derived from PPG signal 500 (FIG. 5A). In some embodiments, PPG signal 500 may be band-pass filtered around a known heart rate to minimize noise and other undesirable signal components. For example, PPG signal 500 may be band-pass filtered around 0.5 to 1.5 times the heart rate of the subject to generate processed PPG signal 522. At least some of the morphology of PPG signal 500 may be preserved throughout the processing of the signal. Changes in the shape of the pulses, as illustrated by points 516, 518, and 520, may remain present. Further, fluctuations in the frequency and amplitude of the pulses due to respiration may remain in the signal. Baseline shifts in the signal (e.g., depicted in segment 512), as well as fluctuations in the timing between fiducial points of pulses in processed PPG signal 522 may also remain in the signal. One or more aspects of the preserved morphology may enable respiration information to be determined.

In some embodiments, values of a physiological signal (e.g., processed PPG signal 522) may be associated with time-delayed values of the same signal. For example, if the physiological signal is referred to as f(t), values of f(t) at discrete times t may be associated with values of f(t+d), where d is a time delay. The time delay d may be fixed or variable. In some embodiments, the time delay d is a function of a rate or period associated with the physiological signal. For example, when the physiological signal is a PPG signal, the time delay d may be selected to be a fraction (e.g., an eighth, a quarter, three-eighths, or any other suitable fraction) or a multiple of the period associated with pulses in the PPG signal. When the time delay d is a quarter period, the associated values may generally form a circular shape when considered in two-dimensional space.

FIG. 6 shows an illustrative attractor 600 generated from pairs of associated values of a processed PPG signal in accordance with some embodiments of the present disclosure. The associated values may have been selected using a delay of a quarter period. In some embodiments, attractor 600 may be generated by plotting a PPG signal against a time-delayed version of itself. In some embodiments, attractor 600 may be generated from processed PPG signal 522 (FIG. 5B). The x-axis of the plot may be chosen to represent the values of the PPG signal, while the y-axis may be chosen to represent values of the time-delayed version of the same PPG signal. Each point of attractor 600 may represent a pair of associated values of the PPG signal and a time-delayed version of the same PPG signal.

As illustrated, the shape of attractor 600 is generally circular. This may be typical of PPG signals that have low noise and exhibit changes in morphology based on respiration. Each pulse period in the PPG signal is generally represented by one loop in attractor 600. The changes in morphology depicted in FIGS. 5A-B are represented as changes in the loops of attractor 600 (e.g., amplitude variations and frequency variations). It will be understood that aperiodicity, and more complex waveforms will result in more complex attractors that may or may not form closed curves. It will also be understood that attractors generated using different time delays may have different shapes. For example, time delays of an eighth of a period and three-eighths of a period may generate generally oval attractors. Circular and oval attractors may be referred to being open. When attractors collapse onto themselves they may be referred to as being closed. For example, a time delay of a half a period may generate a closed attractor that generally lies alone a line. In some embodiments, a signal may be centered about zero (e.g., by performing a mean subtraction), so that the corresponding attractor is substantially centered about (0, 0). If a signal is not centered about zero, corresponding attractors may be substantially centered about points other than (0, 0). Details regarding generating attractors, and analysis thereof, may be found in the book “Fractals and Chaos: An illustrated Course” by Paul S. Addison, 1997, which is hereby incorporated by reference herein in its entirety.

Attractors such as attractor 600 and pairs of associated values may be processed to determine respiration information. FIG. 9 is a flowchart of illustrative steps for determining respiration information from a physiological signal, in accordance with the present disclosure.

Step 902 may include processing equipment receiving a PPG signal from a physiological sensor, memory, any other suitable source, or any combination thereof. For example, referring to system 300 (FIG. 3), the processing equipment may receive a window of physiological data from signal input 310 (FIG. 3). A sensor associated with signal input 310 may be coupled to a subject, and may detect physiological activity such as, for example, RED and/or IR light attenuation by tissue, using a photodetector. In some embodiments, physiological signals generated by signal input 310 may be stored in memory (e.g., RAM 54 (FIG. 2), QSM 72 (FIG. 2), and/or other suitable memory) after being pre-processed by pre-processor 312. In such cases, step 902 may include recalling data from the memory for further processing. In some embodiments, the processing equipment may filter the PPG signal. For example, the processing equipment may apply a high-pass filter (e.g., having a cutoff frequency below the expected heart rate) to reduce or substantially remove baseline changes and other low-frequency artifacts. In a further example, the processing equipment may apply a low-pass filter (e.g., having a cutoff frequency above the expected heart rate) to reduce or substantially remove higher frequency noise or features. In a further example, the processing equipment may apply a band-pass filter to reduce or substantially remove low and high frequency artifacts and features. The band-pass filter may be adjustable and set to filter the input signal around the pulse rate (e.g., from 0.5 times pulse rate to 1.5 times the pulse rate). In some embodiments, steps 904-908 may be based on a Red PPG signal, IR PPG signal, a derivative thereof, a processed signal derived thereof, or any combination thereof.

Step 904 may include the processing equipment associating values of the received PPG signal with values of a time-delayed version of the same PPG signal to generate pairs of associated values. This may be accomplished, for example, by determining time-delayed values of the PPG signal and associating them with non-delayed values of the same PPG signal to form pairs of associated values. In some embodiments, a first segment of the PPG signal may be identified. The length of the first segment may be any suitable length in time or samples. For example, the length of the first segment may be a multiple of a physiological rate (e.g., heart rate or respiration rate). A second segment of the PPG signal may also be identified. The second segment may have the same length as the first segment, but be shifted in time based on a time delay. The time delay may be fixed or variable. In some embodiments, the time delay may be a quarter period (e.g., where the period corresponds to a physiological rate) or any other fraction or multiple of the period. The time delay may also be selected to be an optimal delay to maximize variation in the pairs of associated values due to respiration. In reference to FIG. 6, attractor 600 may represent pairs of associated values generated in step 904.

Step 906 may include the processing equipment analyzing the pairs of associated values to identify a subset of pairs. In some embodiments, the subset of pairs may be identified as corresponding to a curve when the pairs of associated values are considered in two-dimensional space. The curve may be a line, polynomial of second or higher order, a piecewise curve, any other suitable curve, or any combination thereof. For example, when considered in two-dimensional space, the curve may be a horizontal line, a vertical line, an oblique line, or piecewise combination thereof. In some embodiments, identifying the subset of the associated value pairs corresponding to the curve may include identifying intersections of the associated value pairs and the curve. For example, the associated value pairs nearest the curve may be identified. In a further example, associated value pairs on either side of the curve may be identified, and an interpolated associated value pair coincident with the curve may be determined.

In some embodiments, step 906 may include selecting, or otherwise generating, the curve. In some embodiments, the curve used may depend on the time delay. For example, because an attractor is expected to be generally circular for a time delay of approximately a quarter of a period and generally oval for a time delay of approximately an eighth or three-eighths of a period, the desired curves used for such time delays may be different. In some embodiments, the curve can be applied to an optimal location in the attractor (e.g., where the cycle to cycle spread of the attractor is at a maximum or most likely represents variation due to respiration). In some embodiments, the position and shape of the curve may be determined analytically (from expected PPG morphology) or empirically (from stored subject data).

The identification of the subset of associated pairs is depicted graphically, for example, in FIG. 6. FIG. 6 shows attractor 600 and illustrative curves 602 and 604. Curves 602 and 604 are vertical lines that pass through the bottom and top portions of attractor 600, respectively. While two curves are depicted in FIG. 6, a single curve may be used or more than two curves may be used. In some embodiments, the subset of associated pairs may be identified by determining the pair in each loop of attractor 600 that is closest to the curve. In some embodiments, the subset of associated pairs may be identified by identifying the intersection of each loop of attractor 600 and the curve.

In some embodiments, the processing equipment may identify the subset of pairs using an angle technique. The angle technique may be used, for example, on a PPG signal that is centered about zero. The pairs of associated values for a PPG signal centered about zero, when considered in two-dimensional space, will typically loop around the origin. The angle technique may be used to identify a subset of pairs that generally lie on a line that passes through the origin. In some embodiments, an angle may be determined for each pair of associated values. Identifying pairs of associated values whose angles correspond to a predetermined angle may be accomplished by methods well known in the art, for example, by application of the Eq. 14 as shown below:

$$θ = {tan}^{- 1} \frac{y}{x} , \qquad ( 14 )$$

where θ represents the predetermined angle, y represents the portion of the pair of associated values corresponding to the time-delayed version of the PPG signal, and x represents the portion of the pair of associated values corresponding to the non-delayed version of the PPG signal. By plugging each pair of associated values (i.e., x and y) into the equation and determining if the result is equal to the predetermined angle θ, a subset of the pairs of associated values may be identified. For each loop the pairs of associated values take around the origin the angle technique should be able to identify at least one pair of associated values. In some cases, none of the pairs in a loop may have an angle that equals the predetermined angle. This may occur, for example, when the heart rate is high and/or when the sampling rate is low. Accordingly, in some embodiments the angle technique may identify when the angles of the pairs cross the predetermined angle. When a crossing is identified, the pair having an angle closest to the predetermined angle may be identified or an interpolated pair of associated values coincident with the predetermined angle may be identified based on the pairs on either side of the predetermined angle.

In some embodiments, the processing equipment may identify the subset of pairs using a zero crossing technique. The zero crossing technique may be used, for example, on a PPG signal that is centered about zero. The zero crossing technique may be used to identify a subset of pairs that, when considered in two-dimensional space, generally lie on a vertical or horizontal line that passes through the origin. If the pairs of associated values are considered to be in the form (x, y), zero crossings of the x value may correspond to crossings of a vertical line through the origin and zero crossings of the y value may correspond to crossings of a horizontal line. In some embodiments, a rotation operation may be performed on the pairs of associated values before performing the zero crossing technique. By using a rotation operation first, a subset of pairs can be identified that correspond to pairs of associated values that generally lie on a line of any angle that passes through the origin.

It will be understood that the foregoing techniques for identifying a subset of pairs in step 906 is merely illustrative and any suitable techniques for obtaining a slice of an attractor may be used.

Step 908 may include the processing equipment determining respiration information based on the identified subset of pairs. In some embodiments, one or more respiration metrics may be determined from the subset of pairs. A respiration metric may correspond to a single value or multiple values. The respiration metrics may, for example, include one or more amplitude values associated with the subset of pairs, one or more time values associated with the subset of pairs, any other suitable metrics associated with the subset of pairs, and any combination thereof. The one or more respiration metrics may be processed to obtain respiration information, such as respiration rate.

In some embodiments, the amplitude values of the respiration metric may represent the distances from an origin to each of the subset of pairs. Referring back to FIG. 6 and the subset of pairs identified using curve 604, the amplitude values may computed as the distances between origin 608 and intersections of attractor 600 and curve 604. In this example, because curve 604 is a vertical line aligned with the origin, the distances may be determined to be the “y” values of each subset of pairs. More generally, the amplitude value of a pair may be computed using Eq. 15 as shown below:

Amplitude=\sqrt{(Pₓ−Oₓ)²+(P_{y}−O_{y})²}, (15)

where Pₓ is the “x” value of the pair, P_{y} is the “y” value of the pair, Oₓ is the “x” value of the origin, and O_{y} is the “y” value of the origin. The amplitude values may be indicative of the amplitude modulation of the PPG sign due to respiration.

FIG. 7A shows an illustrative plot 700 of amplitude values in accordance with some embodiments of the present disclosure. The y-axis is in units of amplitude and the x-axis of the plot represents the series of pairs from which the amplitude values were calculated. Plot 700 includes amplitude series 702 and amplitude series 704. In some embodiments, amplitude series 702 corresponds to amplitudes calculated from the subset of pairs identified using curve 604 (FIG. 6) and amplitude series 704 corresponds to amplitudes calculated from the subset of pairs identified using curve 602 (FIG. 6). Amplitude series 704 is plotted as negative amplitudes for purposes of clarity to prevent amplitude series 702 and 704 from overlapping in FIG. 7A. The circles of amplitude series 702 and 704 represent the computed amplitude values. In this example, each amplitude value of amplitude series 702 and 704 represents information from one loop of attractor 600 (FIG. 6), which corresponds to one pulse period of the original PPG signal. The amplitude modulation of amplitude series 702 and 704 may represent the amplitude modulation of pulses due to respiration.

In some embodiments, the time values of the respiration metric may represent the time differences between the subset of pairs. Referring back to FIG. 6 and the subset of pairs identified using curve 604, the time values may correspond to time differences between subsequent intersections of attractor 600 and curve 604. The time values may be computed in units of samples, time, or any other suitable units. When the processing equipment identifies the subset of pairs, the processing equipment may store the sample numbers or times associated with the identified pairs and use the stored information to compute the time differences.

FIG. 7B shows an illustrative plot 720 of time values in accordance with some embodiments of the present disclosure. The y-axis is in units of time and the x-axis of the plot represents the series of pairs from which the time values were calculated. Plot 720 includes time series 722 and time series 724. In some embodiments, time series 722 corresponds to time differences calculated from the subset of pairs identified using curve 604 (FIG. 6) and amplitude series 724 corresponds to time differences calculated from the subset of pairs identified using curve 602 (FIG. 6). The circles of time series 722 and 724 represent the computed time differences. In this example, the time differences of time series 722 correspond to the amount of time between consecutive crossings of attractor 600 (FIG. 6) and curve 604 (FIG. 6) and the time differences of time series 722 correspond to the amount of time between consecutive crossings of attractor 600 (FIG. 6) and curve 604 (FIG. 6). The modulation of time series 722 and 724 may represent the frequency modulation of pulses due to respiration.

Referring back to step 908 of FIG. 9, the processing equipment may use one or more of the respiration metrics to determine respiration information. For example, the processing equipment may use amplitude values (e.g., amplitude series 702 (FIG. 7A)), time values (e.g., time series 722 (FIG. 7B)), any other suitable respiration metric values, and any suitable combination thereof to determine respiration information. In some embodiments, the processing equipment may perform a correlation (e.g., an autocorrelation, cross-correlation, any other suitable correlation, or any combination thereof) to determine respiration rate. For example, the respiration rate may be determined based on a time difference between peaks in the correlation output. In some embodiments, the processing equipment may use any other suitable processing techniques or combinations thereof to determine respiration rate, including, for example, Fourier transform techniques, wavelet transform techniques, and time domain techniques.

FIG. 8A shows an illustrative plot 800 of respiration metric values that may be used to determine respiration information in accordance with some embodiments of the present disclosure. In some embodiments, the respiration metric values of plot 800 may correspond to amplitude values, time values, any other suitable values associated with a subset of pairs of associated values, or any combination thereof. FIG. 8B shows an illustrative plot 820 of a correlation signal generated in accordance with some embodiments of the present disclosure. The correlation signal of plot 820 may be generated, for example, by performing an autocorrelation of the respiration metric values of plot 800 (FIG. 8A). An autocorrelation may be considered a mathematical operation used to compare a signal with past and/or future values of the signal. By time-shifting a signal and correlating the signal with itself, an autocorrelation signal can be generated. Peaks may be associated with relatively high correlation, zeros may be associated with relatively low correlation, and troughs may be associated with relatively high anti-correlation. The time difference between peaks of a correlation signal may correspond to a period associated with the signal used to generate the correlation signal. In some embodiments, when respiration metric values are used to generate the correlation signal, the time value between peaks may correspond to the respiration rate. One or more peaks of the correlation signal may be identified, such as peaks 822, 824, and 826. Peak 826 is the highest peak and may correspond to the signal being correlated with itself with a time shift of zero. By computing the time difference between adjacent peaks, the respiration rate can be determined.

FIG. 10A is a flowchart of illustrative steps for determining respiration information based on respiration metric values in accordance with some embodiments of the present disclosure. Step 1002 may include the processing equipment performing an autocorrelation of respiration metric values (e.g., amplitude values or time values). Step 1004 may include the processing equipment analyzing the autocorrelation result to determine respiration information. For example, peaks in the autocorrelation signal may be identified and the distance between two peaks may be determined. The respiration rate may be determined based on the distance between two peaks.

FIG. 10B is a flowchart of illustrative steps for determining respiratory information based on two sets of respiration metric values in accordance with some embodiments of the present disclosure. Step 1022 may include the processing equipment performing a cross-correlation of two sets of respiration metric values. The two sets may be of the same or different types of respiration metric values. For example, the two sets may be two sets of amplitude values (e.g., amplitude series 702 (FIG. 7A) and amplitude series 704 (FIG. 7A)), two sets of time values (e.g., time series 722 (FIG. 7B) and time series 724 (FIG. 7B)), one set of amplitude values and one set of time values (e.g., amplitude series 702 (FIG. 7A) and time series 722 (FIG. 7B)), or any other combination of respiration metric values. A cross-correlation may be considered a mathematical operation used to compare two different signals. By time-shifting a first signal relative to a second signal and correlating values of the first signal with values of the second signal, a cross-correlation signal can be generated. Peaks may be associated with relatively high correlation, zeros may be associated with relatively low correlation, and troughs may be associated with relatively high anti-correlation between the two signals. Step 1024 may include the processing equipment analyzing the cross-correlation result to determine respiration information. In some embodiments, the processing equipment may perform the same analysis described in connection with step 1004 (FIG. 10A)

FIG. 10C is a flowchart of illustrative steps for determining respiration information based on two sets of respiration metric value. Step 1042 may include the processing equipment performing an autocorrelation of a first set of respiration metric values. In some embodiments, the first set of respiration metric values may be amplitude values, time values, any other suitable respiration metric values, or any combination thereof. Step 1044 may include the processing equipment performing an autocorrelation of a second set of respiration metric values. In some embodiments, the second set of respiration metric values may be amplitude values, time values, any other suitable respiration metric values, or any combination thereof. Step 1046 may include the processing equipment combining the results from steps 1042 and 1044 and analyzing the combined results to determine respiration information. In some embodiments, the results of steps 1042 and 1044 may be combined by summing, averaging, or performing a weighted average. In some embodiments, the analysis performed by the processing equipment may be the same analysis described in connection with step 1004 (FIG. 10A).

In view of the foregoing, it will be understood that the processing equipment in step 908 of FIG. 9 may perform one or more of the flowcharts of FIGS. 10A-C, or any other suitable techniques, to determine respiration information. It will also be understood that while step 908 has been described as determining respiration rate, any other suitable respiration information may be determined. For example, the phase of the respiration metric values may be analyzed to determine the timing of individual breaths. In addition, the amount of modulation of the respiration metric values may be used to determine respiratory effort. Once the respiration information is determined, the respiration information may be averaged with previously determined respiratory information and outputted for display on, for example, display 20 of FIGS. 1-2.

The foregoing is merely illustrative of the principles of this disclosure and various modifications may be made by those skilled in the art without departing from the scope of this disclosure. The above described embodiments are presented for purposes of illustration and not of limitation. The present disclosure also can take many forms other than those explicitly described herein. Accordingly, it is emphasized that this disclosure is not limited to the explicitly disclosed methods, systems, and apparatuses, but is intended to include variations to and modifications thereof, which are within the spirit of the following claims.

What is claimed:

1. A method for determining respiration information, the method comprising:
receiving a photoplethysmograph (PPG) signal;
associating values of the PPG signal with time delayed values of the PPG signal to generate pairs of associated values;
analyzing the pairs of associated values to identify a subset of the pairs; and
determining respiration information based at least in part on the subset of the pairs.

2. The method of claim 1, wherein the PPG signal is approximately centered about zero.

3. The method of claim 1, further comprising:
receiving heart rate information; and
selecting a time delay of the time delayed values of the PPG signal to be approximately one quarter of a period associated with the heart rate information.

4. The method of claim 1, wherein analyzing the pairs of associated values to identify a subset of the pairs comprises identifying pairs of associated values that approximately form a straight line when the pairs are considered in two-dimensional space.

5. The method of claim 1, wherein analyzing the pairs of associated values to identify a subset of the pairs comprises:
determining angles corresponding to the pairs of associated values; and
identifying pairs of associated values whose angles correspond to a predetermined angle.

6. The method of claim 1, wherein analyzing the pairs of associated values to identify a subset of pairs comprises identifying zero crossings associated with the pairs of associated values.

7. The method of claim 1, wherein determining respiration information comprises determining one or more respiration metric values based on the subset of the pairs.

8. The method of claim 7, wherein the one or more respiration metric values are one or more of amplitude values and time values corresponding to the subset of pairs.

9. The method of claim 7, wherein determining respiration information comprises performing a correlation based on the respiration metric values.

10. The method of claim 1, wherein determining the respiration information comprises determining respiration rate.

11. A physiological monitoring system, the system comprising:
processing equipment configured to:
receive a photoplethysmograph (PPG) signal;
associate values of the PPG signal with time delayed values of the PPG signal to generate pairs of associated values;
analyze the pairs of associated values to identify a subset of the pairs; and
determine respiration information based at least in part on the subset of the pairs.

12. The system of claim 11, wherein the processing equipment is further configured to approximately center the PPG signal about zero.

13. The system of claim 11, wherein the processing equipment is further configured to:
receive heart rate information; and
select a time delay of the time delayed values of the PPG signal to be approximately one quarter of a period associated with the heart rate information.

14. The system of claim 11, wherein the processing equipment is configured to identify the subset of pairs by identifying pairs of associated values that approximately form a straight line when the pairs are considered in two-dimensional space.

15. The system of claim 11, wherein the processing equipment is configured to identify the subset of pairs by:
determining angles corresponding to the pairs of associated values; and
identifying pairs of associated values whose angles correspond to a predetermined angle.

16. The system of claim 11, wherein the processing equipment is configured to identify the subset of pairs by identifying zero crossings associated with the pairs of associated values.

17. The system of claim 11, wherein the processing equipment is configured to determine the respiration information by determining one or more respiration metric values based on the subset of pairs.

18. The system of claim 17, wherein the one or more respiration metric values are one or more of amplitude values and time values corresponding to the subset of pairs.

19. The system of claim 17, wherein the processing equipment is configured to determine respiration information by performing a correlation based on the respiration metric values.

20. The system of claim 11, wherein the respiration information comprises respiration rate.
